(19) United States
(12) Patent Application Publication
(10) Pub. No.: US 2009/0018583 A1
(43) Pub. Date: Jan. 15, 2009

(54) Dynamic spinal stabilization system incorporating a wire rope

(75) Inventors: John K. Song, Chicago, IL (US); Jeffrey David Gordon, Saratoga Springs, NY (US)

Correspondence Address: JOHN K. SONG, 474 NORTH LAKE SHORE DR., APT. # 4508, CHICAGO, IL, 60611-6465, US

(73) Assignee: Vermillion Technologies, LLC, Chicago, IL (US)

(21) Appl. No.: 11/827,423

(22) Filed: Jul. 12, 2007

(51) Int. Cl. A61B 17/70; A61B 17/56; A61B 17/04; A61B 17/58

(52) U.S. Cl. 606/246; 606/100; 606/263; 606/264; 606/301

(57) ABSTRACT

A device, or series of devices, for use in stabilizing two or more vertebrae of the spine—specifically, a wire rope which is used with pedicle screws as a dynamic spinal stabilization system, thereby allowing limited motion of the vertebrae. The system may also allow load transfer to an intervertebral bone graft or bone graft substitute.

### FIELD OF THE INVENTION

The present invention describes a surgical device incorporating a wire rope which is meant to stabilize at least one vertebra relative to another vertebra while transmitting loads and/or motion between the spinal structures.

### BACKGROUND OF THE INVENTION

Low back pain is one of the most expensive diseases afflicting industrialized societies. With the exception of the common cold, it accounts for more doctor visits than any other ailment. The spectrum of low back pain is wide, ranging from periods of intense disabling pain which resolve, to varying degrees of chronic pain. The conservative treatments available for lower back pain include: cold packs, physical therapy, narcotics, steroids and chiropractic maneuvers. Once a patient has exhausted all conservative therapies, the surgical options range from micro-discectomy, a relatively minor procedure relieving pressure on the nerve root and spinal cord, to fusion which eliminates spinal motion at the level of pain.

Each year, over 200,000 patients undergo lumbar fusion surgery in the United States. While fusion is effective about seventy percent of the time, there are consequences even to these successful procedures, including a reduced range of motion and an increased load transfer to adjacent levels of the spine, which may accelerate degeneration at those levels. Further, a significant number of back-pain patients, estimated to exceed seven million in the U.S., simply endure chronic low-back pain rather than risk procedures that may not be appropriate or effective in alleviating their symptoms.

New treatment devices, collectively called motion preservation devices, are currently being developed to address these limitations. Some promising therapies include nucleus, disc or facet replacements. Other motion preservation devices provide dynamic internal stabilization of the injured and/or degenerated spine, without removing any spinal tissues. The goal of these devices is to stabilize the spine to prevent pain while preserving near normal spinal function.

U.S. Pat. No. 6,290,700 to Schmotzer describes a device in which tension force is exerted on pedicle screws with a tensioning wire while bumpers maintain the distance between the screws. The tensioning wire resists elongation of the stabilizing device and the bumper resists compression. The entire stabilizing device stiffens the spine and may correct deformity or misalignment.

U.S. Pat. Nos. 6,986,771 and 6,989,011 to Paul describe many dynamic spinal stabilization devices with significantly differing constructions, including one embodiment incorporating a “braided wire”.

U.S. Pat. No. 7,137,985 to Jhang also describes many dynamic spinal stabilization devices with significantly differing constructions, one embodiment incorporating wires “interweaved or braided together to form a braided metal wire rod”. Also, the patent includes “metal strips, strands or ribbons interweaved in a diagonally overlapping pattern”.

### SUMMARY OF THE INVENTION

The present invention describes a surgical device which is meant to stabilize at least one vertebra relative to another vertebra with a compliant device capable of transmitting loads and/or motion between spinal structures. The invention takes advantage of wire rope technology which has been successfully utilized for countless engineering applications. Machinery's Handbook, a well known and respected reference for mechanical devices, defines wire rope as follows: “Essentially, a wire rope is made up of a number of strands laid helically about a metallic or non-metallic core. Each strand consists of a number of wires also laid helically about a metallic or non-metallic center” [1]. In analysis of the wire rope and braided wire (or braided cable) literature, it is apparent that wire rope (also known as twisted rope or laid rope) is distinctly different from “braided wire”, “braided cable” or “braided rope” in both construction and aggregate properties. Braided wire is manufactured by weaving strands over and under each other in a crossing manner. Braided wire has been utilized in limited applications, but wire rope has a long history of successful use. The current invention utilizes the above mentioned definition of wire rope from Machinery's Handbook with no restriction on the direction of the helical path of the individual wires or the strands, or the number, size or pattern of the individual wires or the strands. In fact, a benefit of the current invention is that the mechanical properties of the wire rope, such as flexibility, abrasion resistance, resistance to unwinding, torsional rigidity and strength can be controlled by the lay of the wire rope (the direction of the helical path of the individual wires and the strands) and the construction of the wire rope (the diameter, number and pattern of the individual wires and the strands).

It is an object of the present invention to provide a flexible surgical implant means for stabilizing two adjacent vertebrae.

It is a further object of the present invention to provide a surgical implant for spinal fusion surgeries in which a bone graft, bone substitute or other spacer which may contain bone graft or bone substitute, is implanted into the intervertebral space between the adjacent vertebrae.

It is a further object of the present invention to provide a surgical implant means for stabilizing two or more non-adjacent vertebrae.

It is a further object of the present invention to allow loads to be borne by the above mentioned bone graft or bone graft substitute. Bearing these loads has been shown to be beneficial to the healing or consolidation of the graft and incorporation of the graft material with the apposite vertebrae.

It is a further object of the present invention to allow limited flexion and extension bending motion of the vertebrae to which it is attached.

It is a further object of the present invention to allow limited axial motion of the vertebrae to which it is attached, thereby allowing loading of the disc space.

It is a further object of the present invention to allow limited lateral bending motion of the vertebrae to which it is attached.

It is a further object of the present invention to limit or prevent axial rotation and anterior/posterior translational motion of the vertebrae to which it is attached.

### BRIEF DESCRIPTION OF THE DRAWINGS

FIG. 1A shows a wire rope

FIG. 1B is a detail view of a wire rope showing the rope's construction

FIG. 2 shows various wire rope constructions

FIG. 3 is a perspective view of two adjacent vertebrae with the invention implanted.

FIG. 4 is an exploded perspective view of the preferred embodiment of the invention with two (2) pedicle screws for stabilizing adjacent vertebrae

FIG. 5 is a perspective view of the preferred embodiment of the invention with two (2) pedicle screws for stabilizing adjacent vertebrae

FIG. 6 is a perspective view of an alternative embodiment of the invention with rigid and flexible portion and three (3) pedicle screws for stabilizing three vertebrae

FIG. 7 is an exploded perspective view of an alternative embodiment of the invention

FIG. 8 is a perspective view of an alternate embodiment of the invention

FIG. 9 is a perspective view of an alternate embodiment of the invention used for augmenting a fusion system.

FIG. 10 is an exploded perspective view of an alternate embodiment of the device used for augmenting a fusion system

FIG. 11 is a perspective view of an alternate embodiment of the device used for augmenting a fusion system

FIG. 12 is an exploded view of an alternative embodiment of the invention

FIG. 13 is an alternative embodiment of the invention

FIG. 14 is a perspective view of the wire rope with the preferred adapting means attached to the end

FIG. 15 shows the wire rope with the preferred adapting means attached to the end.

FIG. 16 shows an alternative embodiment of the adapting means

FIG. 17 shows an alternative embodiment of the adapting means

FIG. 18 shows an alternative embodiment of the adapting means

FIG. 19 shows an alternative embodiment of the adapting means

### DETAILED DESCRIPTION OF THE DRAWINGS

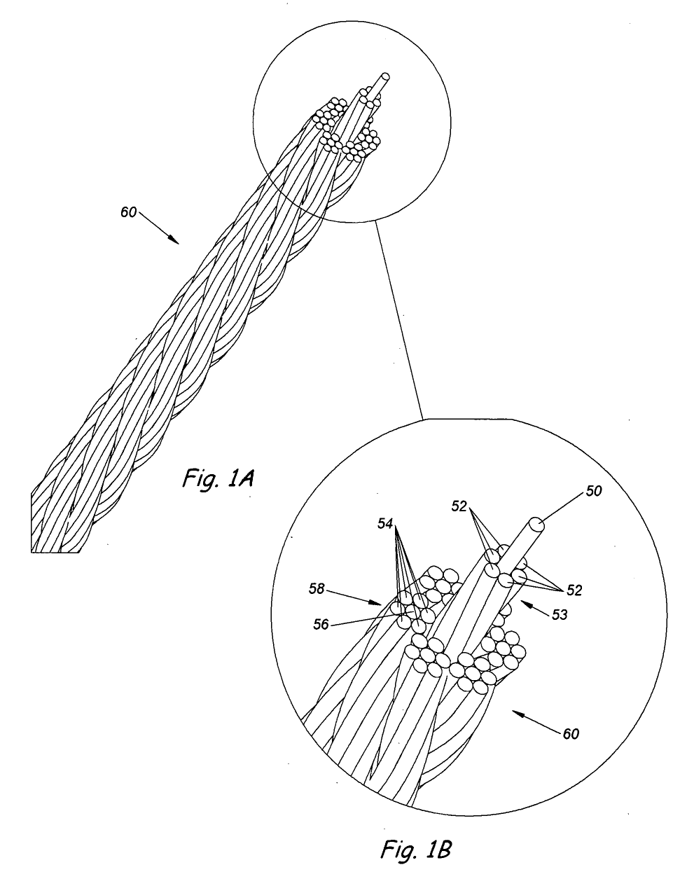

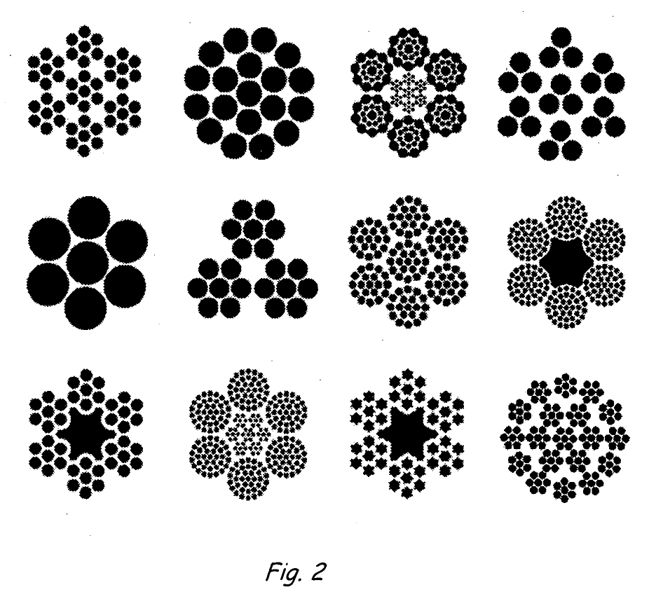

FIGS. 1A and 1B show a wire rope. Wires 52 are twisted around a core wire 50 in a helical manner to create a first strand 53 of a multi-strand wire rope 60. In the same way, wires 54 are twisted around another core 56 in a helical manner and the resulting strand 58 is twisted around the first strand 53 in a helical manner. Multiple other strands are wrapped around first strand 53 in a helical manner until wire rope 60 is created as shown. This describes just one method of creating a wire rope. In general, the inventors define a wire rope as being made up of a number of strands laid helically about a metallic or non-metallic core; each strand consisting of a number of wires also laid helically about a metallic or non-metallic center. FIG. 2 shows some examples of typical wire rope constructions. This figure is not meant to be an exhaustive list of possible constructions since there are many possible constructions which would serve the purpose of this invention.

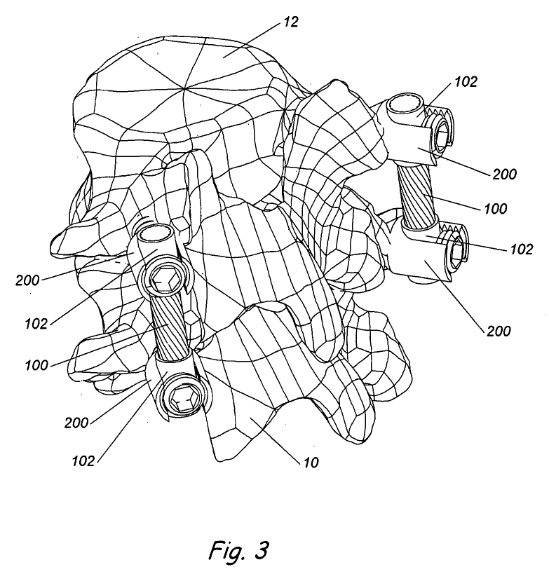

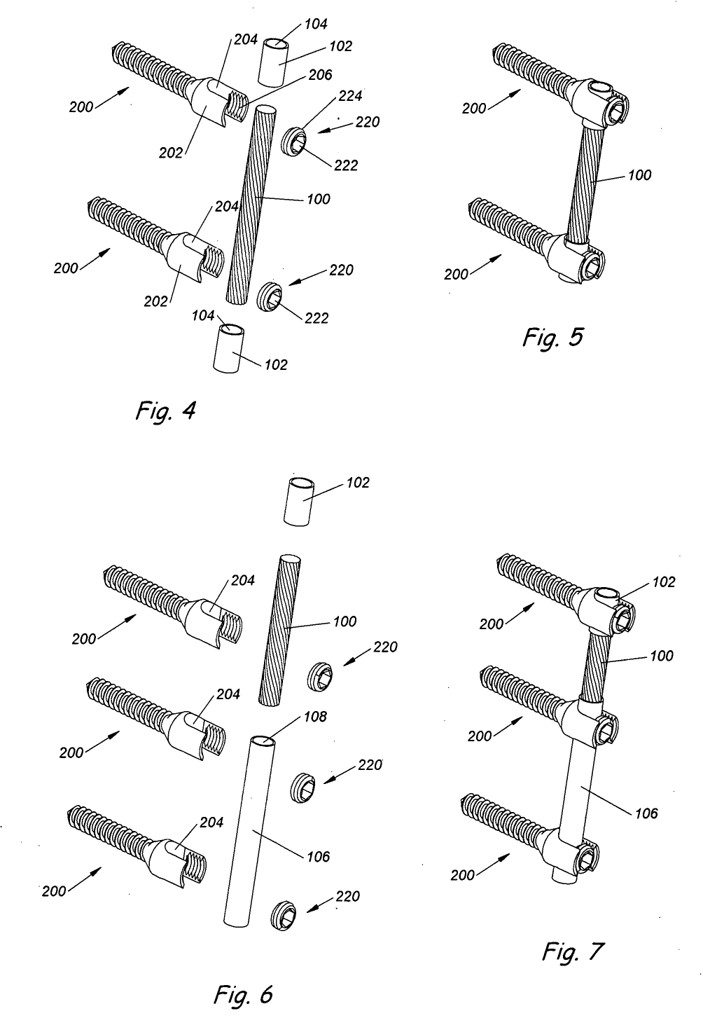

FIGS. 3, 4 and 5 show the preferred embodiment of the invention. FIG. 3 shows a first vertebra 10, a second vertebra 12, and the intervertebral disc between the vertebrae, together called a “functional spinal unit” or FSU, with the invention attached to both vertebrae. Two pedicle screws 200 are inserted into the pedicles of each of the first vertebra 10 and second vertebra 12. A preferred embodiment of the invention, consisting of two cylindrical collars 102 attached to a wire rope 100 by welding, swaging, crimping, press-fit, shrink-fit, bonding, some other attachment means or simply by slip fit, is inserted into a recess 204 (see FIG. 4) in each of the pedicle screws. A set screw 220 with threads 224 is threaded into threads 206 in head 202 of each pedicle screw 200 to rigidly attach wire rope 100 to pedicle screw 200. Set screw 220 may also deform cylindrical collar 102 to crimp it onto wire rope 100. The resulting construct is meant to give the spinal level pliable stabilization, i.e. to provide mechanical resistance to spinal motion.

FIGS. 6 and 7 show an alternative embodiment of the invention. In some instances it may be necessary to apply rigid stabilization to at least one FSU and flexible stabilization to at least one other adjacent FSU. A rigid stabilization rod 106 with a cavity 108 has one end of a wire rope 100 attached within cavity 108 by welding, swaging, crimping, press-fit, shrink-fit, bonding, some other attachment means or simply by slip fit. A cylindrical collar 102 is attached to the other end of wire rope 100 by one of the aforementioned means. A set screw 220 is screwed into each pedicle screw 200 as described above to attach each wire rope 100 and each rigid stabilization rod 106 to a vertebra.

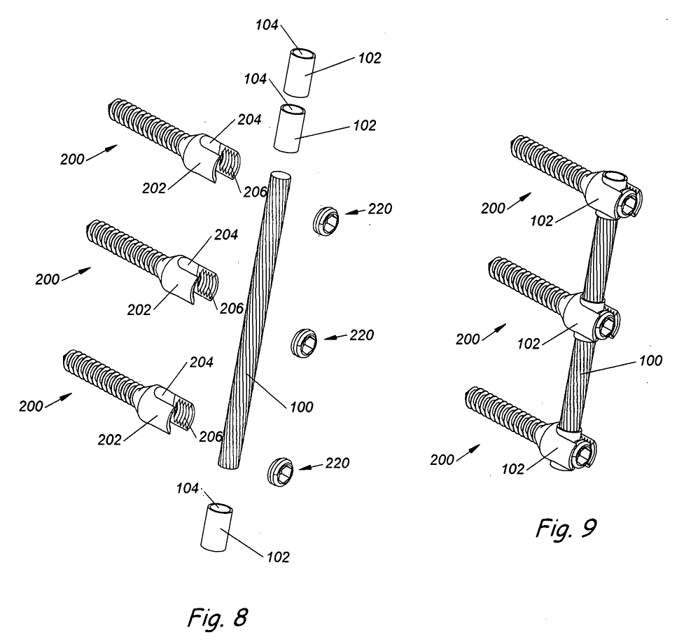

FIGS. 8 and 9 show a further alternative embodiment of the invention. In some instances it might be necessary to apply flexible stabilization to more than one FSU. A wire rope 100 has multiple cylindrical collars 102 attached to it by welding, swaging, crimping, press-fit, shrink-fit, bonding, some other attachment means or simply by slip fit. A set screw 220 is threaded into each pedicle screw 200 as described above to attach each wire rope 100 to the vertebrae.

FIGS. 10 and 11 show a further alternative embodiment of the invention. In some instances it might be beneficial to apply flexible stabilization to at least one FSU adjacent to a standard rigid stabilization rod 250. A collet 300 contains a body portion 302 with a first recess 304 and a threaded portion 305 with slits 306 and a second recess 308. A wire rope 100 is attached to collet 300 by inserting one end into first recess 304 and welding, swaging, crimping, press-fit, shrink-fit, bonding, some other attachment means or simply by slip fit. Collet 300 is attached to standard rigid stabilization rod 250 by slipping standard rigid stabilization rod 250 into second recess 308 and clamping collet 300 with a collet clamping nut 320 which has flats for engagement with a wrench (not shown). At least one cylindrical collar 202 is attached as described above. A set screw 220 is threaded into each pedicle screw 200 as described above to attach each wire rope 100 and each standard rigid stabilization rod 250 to vertebrae.

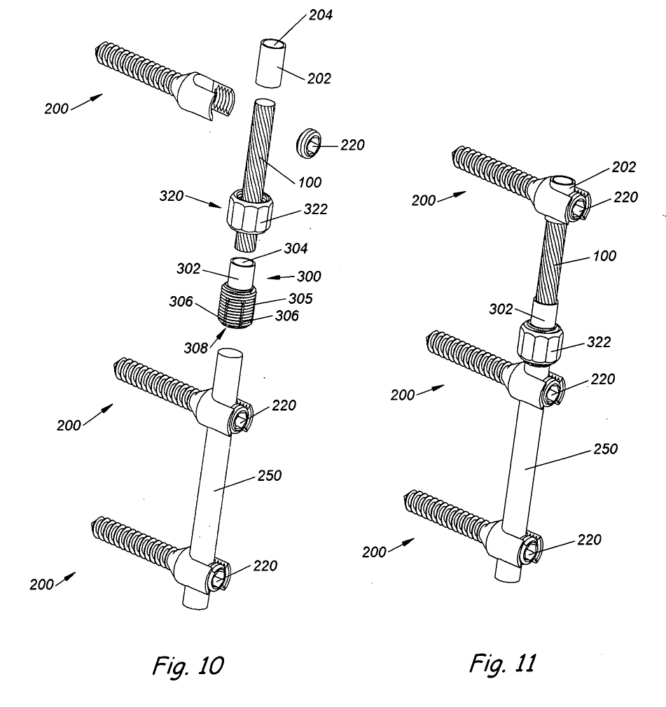

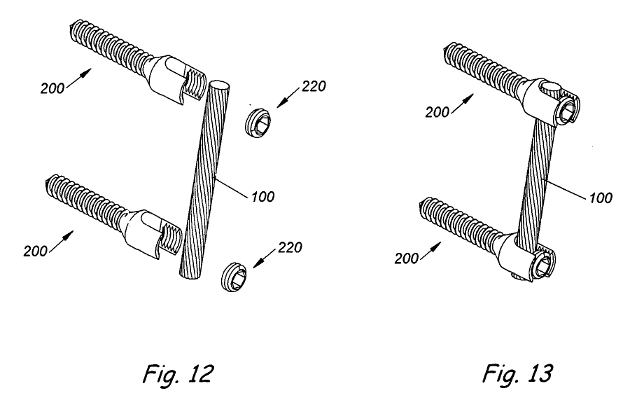

FIGS. 12 and 13 show a further alternative embodiment of the invention where a wire rope 100 is attached directly to pedicle screws 200 with set screws 220.

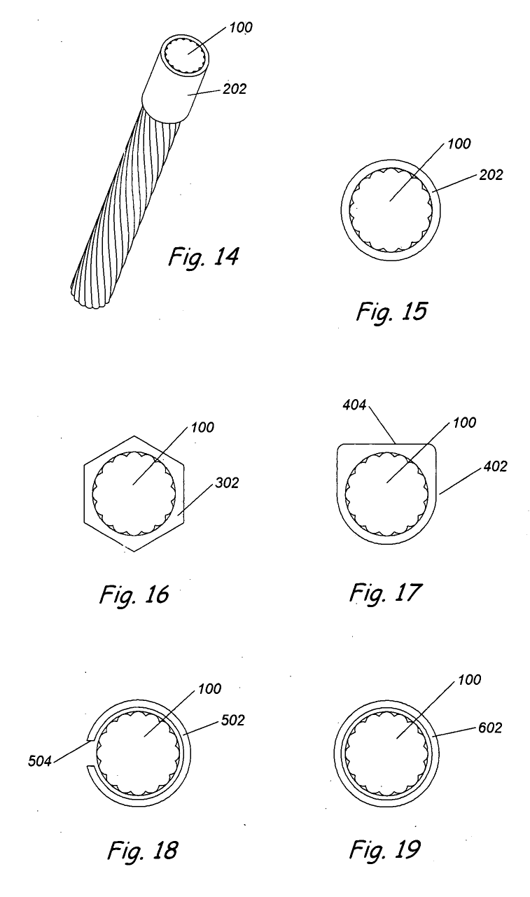

FIGS. 14 through 19 show some alternative embodiments of collars used to attach a wire rope 100 to a vertebra. FIGS. 14 and 15 show a cylindrical collar 202 which has been attached to wire rope 100 by welding, swaging, crimping, press-fit, shrink-fit, bonding, some other attachment means or simply by slip fit. FIG. 16 shows a hexagonal collar 302 which has been similarly attached to a wire rope 100. The advantage of hexagonal collar 302 is that set screw 220 engages hexagonal collar 302 on a flat surface and therefore prevents rotation of wire rope 100. FIG. 17 shows a u-shaped collar with a flat surface 404 for engagement with set screw 220. FIG. 18 shows a split cylindrical collar 502. When acted on by set screw 220, the diameter of split cylindrical collar 502 will reduce and compress onto wire rope 100. Similarly, FIG. 19 shows a cylindrical oversized collar 602 which when acted apon by set screw 220 will collapse onto wire rope 100.

Many pedicle screw designs have been utilized for spinal surgery. The references in this document to set screws can easily be replaced by other types of attachment means incorporated by other pedicle screw designs meant to attach the screws to rods. Therefore this invention should not be limited by the pedicle screw design presented in the figures.

What is claimed is:

1. A device for spinal surgery incorporating:
a stabilization member which can be connected to one or more fixation members which are each attached to the pedicle or lateral mass of at least one vertebra of the spine;
said stabilization member comprising at least one wire rope.

2. The device of claim 1 wherein said fixation member is a bone screw.

3. The device of claim 1 wherein said fixation member is a pedicle screw.

4. The device of claim 1 wherein said fixation member is a lateral mass screw.

5. The device of claim 1 wherein said fixation member is a laminar clamp.

6. The device of claim 1 wherein said stabilization member incorporates at least one adaptation feature for mating with said fixation members.

7. The device of claim 6 where the at least one adaptation feature is a cylindrical ferrule.

8. The device of claim 6 where the at least one adaptation feature is employed by the action of attachment to said fixation member.

9. The device of claim 1 wherein said stabilization member further comprises an adaptation means for attachment to another stabilization member or a rigid rod.

10. The device of claim 9 wherein said attachment means is a collet.

11. The device of claim 9 wherein said attachment means is a clamp.

12. A device for stabilization of the bones of the spine comprising a number of fixation members mated to one or more vertebrae of the spine and a stabilization member connecting said fixation members where the stabilization member comprises 2 or more lengths of biocompatible material twisted together such that said lengths are arranged so that their paths do not substantially interweave or cross.

13. The device of claim 12 where said lengths are twisted in substantially the same direction.

14. The device of claim 12 where said lengths are twisted in substantially different directions.

15. The device of claim 12 where said lengths are twisted in substantially parallel directions.

16. The device of claim 12 wherein said fixation member is a bone screw.

17. The device of claim 12 wherein said fixation member is a pedicle screw.

18. The device of claim 12 wherein said fixation member is a lateral mass screw.

19. The device of claim 12 wherein said fixation member is a laminar clamp.

20. The device of claim 12 wherein said stabilization member incorporates at least one adaptation feature for mating with said fixation members.
